(19) United States
(12) Patent Application Publication
(10) Pub. No.: US 2015/0335472 A1
(43) Pub. Date: Nov. 26, 2015

(54) THERMAL ASSISTANCE DEVICES, OUTERWEAR GARMENTS AND HEATING OR COOLING DEVICES FOR SAID THERMAL ASSISTANCE DEVICES

(71) Applicant: Qore Performance, Inc., Fairfax, VA (US)

(72) Inventors: Justin B. LI, Fairfax, VA (US); J. D. WILLCOX, Fairfax, VA (US); Scott B. STERN, Fairfax, VA (US)

(21) Appl. No.: 14/816,358

(22) Filed: Aug. 3, 2015

Related U.S. Application Data
(63) Continuation-in-part of application No. 13/904,329, filed on May 29, 2013.

(51) Int. Cl. A61F 7/02

(52) U.S. Cl. CPC A61F 7/02; A61F 2007/004

(57) ABSTRACT

A thermal assistance device is useful to provide supplemental heating or cooling to a user's circulating core blood, or to the blood supply to an extremity of a user. Various thermal assistance devices may hold one or more heating or cooling devices or may be covered by an outerwear garment that also provides access to the one or more heating or cooling devices.

### CROSS REFERENCE TO RELATED APPLICATION

This application is a continuation-in-part of U.S. application Ser. No. 13,904,329, filed on May 29, 2013, the entirety of which is incorporated herein by reference.

### BACKGROUND

1. Field of the Invention

The present invention relates to devices useful for providing supplemental heating or cooling to a person's circulating core blood. The devices may be configured as a garment, or as a band, strap, or wrap, for example, to be worn on a wrist, arm, knee, or leg. The thermal assistance devices may also be configured in a variety of other forms such as e.g., pants, shorts, a shirt, jacket, etc. The inventive devices or garments may also be used to provide supplemental heating or cooling to the blood supply to a person's extremity (i.e., to a hand or foot, for example). The thermal assistance devices may also incorporate unique heating or cooling devices and/or may be covered by an outwear garment/shell providing access to the heating or cooling devices without removing the outerwear garment.

2. Related Technology

People involved in strenuous activities, especially in hot climates, or possibly in protective or restrictive clothing and apparel are at risk of overheating (hyperthermia). Overheating reduces a person's mental faculties, reduces stamina, and generally compromises physical, and mental faculties, and the ability to accomplish tasks. On the other hand, people in cold conditions are at risk of hypothermia. Again, hypothermia reduces a person's physical stamina, compromises mental faculties, and reduces the ability to accomplish tasks. An over-arching consideration is that human beings have a limited range of working temperatures in which they can function effectively. Both high or low ambient temperature conditions, heavy or restrictive clothing, heavy physical exertions, or the task of accomplishing complex or demanding tasks, all can contribute to a too high or too low of a core body temperature. People who experience either too high or too low a core body temperature experience compromised performance.

Human extremities (i.e., the hands and feet, especially if exposed) are susceptible to loss of function or dexterity as a result of environmental cold, especially in conditions of extreme cold or conditions of rapid heat loss from the hands, such as immersion in water. Under such conditions if a person's hands lose too much heat and become chilled the person loses hand dexterity, strength, and precision of movement. Similarly, if a person's feet lose too much heat in extreme conditions frostbite of the toes and feet, and catastrophic damage or loss of the toes or feet can result.

Historically, people have attempted to deal with excessive heat by expedients such as forehead absorptive or cooling bands, damp handkerchiefs about the neck, and similar. Excessive cold has been dealt with by gloves, insulted boots, catalytic combustion hand warmers, and similar. None of these historical expedients has been entirely satisfactory.

### SUMMARY

Accordingly, one particularly preferred embodiment of the present invention provides a thermal assistance device comprising an especially configured holder disposed at a selected location on a wearer's body. The especially configured holder is configured for receiving and disposing a heating or cooling device in an abiding heat transfer relation with the wearer's skin at the selected location. The especially configured holder is disposed at a selected location where there is a superficially exposed, or near-superficially exposed (i.e., a pulse point) artery, and mild compression is maintained between the heating or cooling device and the wearer's skin, so as a result an effective heat transfer relationship with the wearer's circulating blood is established and maintained between the heating or cooling device and the person's circulating blood.

Other embodiments disclosed herein provide a unique heating or cooling device, and a thermal assistance device having properly configured holders for such heating or cooling devices, designed to provide heating or cooling for specific pulse points or other portions of the wearer's body that may become exposed during athletic or other movements. In one embodiment, the unique heating or cooling device has a shape similar to a “roll of quarters” and is designed to address superficial exposures of the popliteal artery at the back of the wearer's knees and/or the femoral artery at the front of the wearer's hips.

Other embodiments disclosed herein provide a unique vacuum sealed heating or cooling device configured to be activated out in the field, yet remain silent until activated, which is particularly useful when the properties of the heating or cooling device are not required until a specific point in time and where insulation and active cooling mediums are not available (e.g., for military and police operations, hiking, backpacking, search and rescue, disaster relief, HAZMAT, infectious disease response, etc.).

Other embodiments disclosed herein provide outer garments that can be worn over any thermal assistance device disclosed herein. The outer garment includes regions for accessing the holders containing the heating or cooling devices held within the thermal assistance device so that the heating or cooling devices can be accessed, removed, changed, activated, etc. through the outer garment.

Other objects, features, and advantages of the present invention will be apparent to those skilled in the art from a consideration of the following detailed description of a preferred exemplary embodiment thereof taken in conjunction with the associated figures which will first be described briefly.

Embodiments of the invention relate to such thermal assistance devices having especially configured pockets, holders, or receptacles disposed at one or more selected locations of a wearer's body. A heating or cooling device held by the thermal assistance devices is disposed in an abiding heat transfer relation of mild compression with the person's skin, and in a resulting effective heat transfer relationship with the person's circulating blood in superficially exposed, or near-superficially exposed (i.e., a pulse point) artery. Preferably, both the especially configured pocket, holder, or receptacle, and the heating or cooling device are both elongate, are disposed over the superficially exposed artery, and are aligned along their length with this artery. By providing supplemental cooling or heating of the circulating blood, the person's core body temperature can be moderated toward a desired temperature range. Alternatively, supplemental heating or cooling to a person's extremity may be accomplished.

### BRIEF DESCRIPTION OF THE DRAWINGS

FIG. 1 is a diagrammatic view of a person wearing a garment embodying an example embodiment of the present invention;

FIG. 2 is a fragmentary cross sectional view taken along line 2-2 of FIG. 1, and viewing in the direction of the arrows;

FIG. 3 is a fragmentary cross sectional view taken along line 3-3 of FIG. 1, and viewing in the direction of the arrows;

FIG. 4 is a diagrammatic view of a person wearing an alternative embodiment of a garment embodying the present invention;

FIG. 5 is a diagrammatic view similar to FIG. 1 but showing a much smaller portion of a person wearing an alternative embodiment of a garment embodying the present invention;

FIG. 6 is a fragmentary cross sectional view taken along line 6-6 of FIG. 5, and viewing in the direction of the arrows;

FIG. 7 is a fragmentary view of still another alternative embodiment of the present invention, configured as a strap, band, or wrap to be worn on the wrist, arm or leg of a user;

FIG. 8 illustrates an example embodiment of a heating or cooling device configured to address superficial exposures of e.g., the popliteal artery at the back of the wearer's knees and/or the femoral artery at the front of the wearer's hips;

FIGS. 9 and 10 illustrate example embodiments of garments containing holders for accepting the heating or cooling device illustrated in FIG. 8;

FIG. 11 illustrates an example embodiment of a vacuum sealed heating or cooling device constructed in accordance with the disclosed principles;

12 illustrates another example embodiment of a vacuum sealed heating or cooling device constructed in accordance with the disclosed principles;

FIG. 13 illustrates an example embodiment of an outerwear garment to be provided over a thermal assistance device constructed in accordance with the disclosed principles;

FIG. 14 illustrates another example embodiment of an outerwear garment to be provided over a thermal assistance device constructed in accordance with the disclosed principles;

FIG. 15 illustrates a front view of an example access points to be used with the garments illustrated in FIGS. 13 and 14; and

FIGS. 16-18 illustrate example closure mechanisms for the access panels used with the garments illustrated in FIGS. 13 and 14.

### DEATILED DESCRIPTION

While the present invention may be embodied in many different forms, disclosed herein are several specific exemplary embodiments, which illustrate and explain the principles of the invention. In conjunction with the description of these embodiments, a method of providing supplemental heating and cooling to the circulating blood, or to an extremity of a person will be apparent. It should be emphasized that the present invention is not limited to the specific embodiments illustrated.

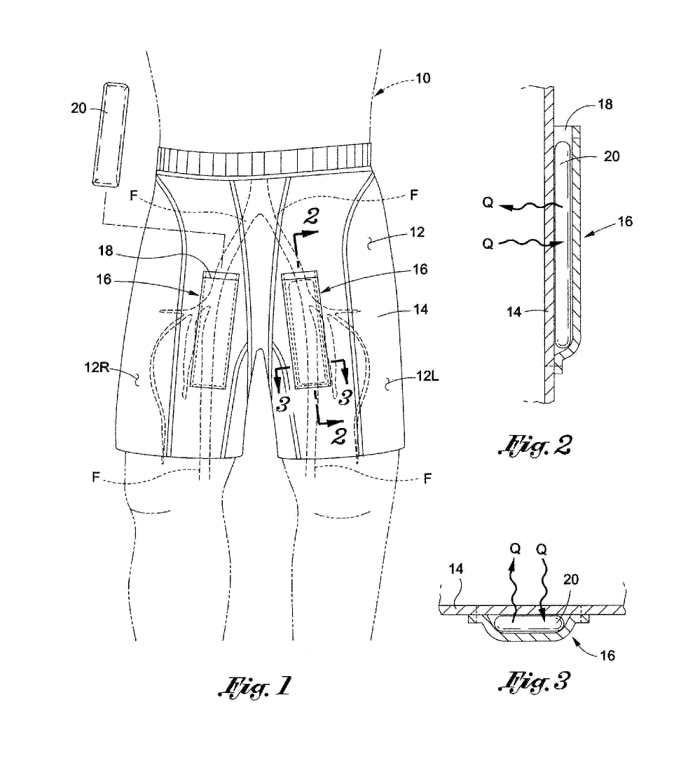

Viewing FIG. 1, a human 10 is depicted using or wearing a thermal assistance garment 12 according to a first embodiment of the present invention. The garment 12 in this embodiment is configured as a pair of compression shorts. Conventional versions of compression shorts will be familiar to joggers, bicycle riders, and others engaged in athletic pursuits. Accordingly, it is seen that the garment 12 includes an elastic outer shell of material 14, which forms the shorts 12, including a waist band 12A, and a left and right leg portion 12L and 12R, respectively. However, in contrast to the conventional compression shorts, the thermal assistance garment 12 according to this invention includes a pair of elongate generally vertically oriented pocket features, generally indicated with the arrowed numerals 16. The pockets 16 are disposed in this embodiment on the outside of the shell material 14, and each defines an upper opening 18. An elongate thermal pack or package 20 is received into each one of the pockets 16, as is indicated on the left-hand side of FIG. 1.

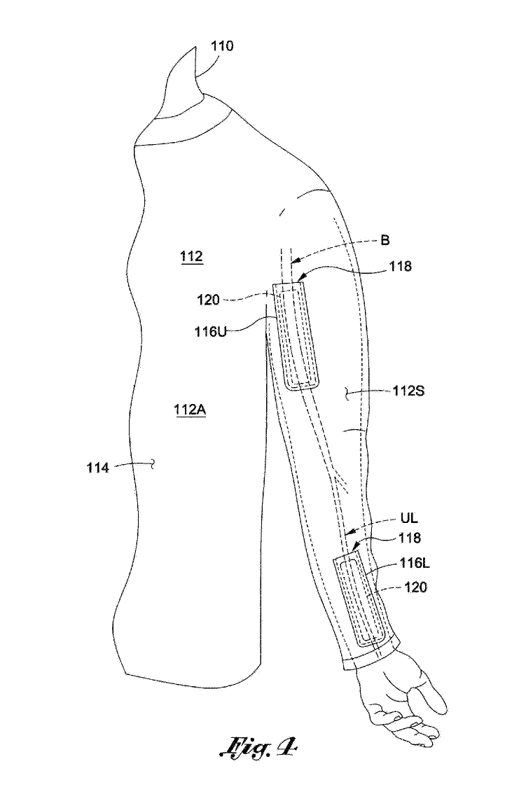

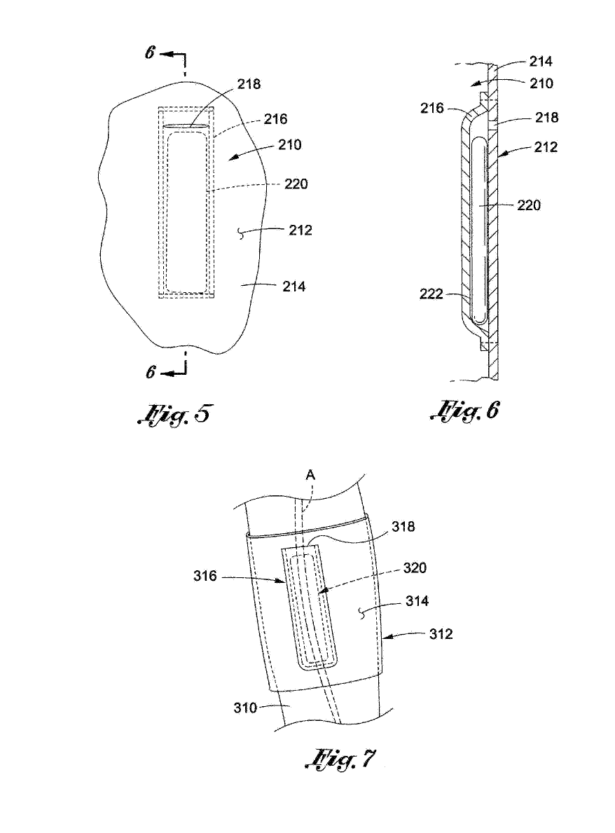

FIGS. 2 and 3 in conjunction indicate that because of the elasticity of the garment 12, the thermal packs 20 are disposed immediately adjacent to and in heat transfer relationship with the skin of the wearer 10, as is indicated by the arrowed characters “Q” on FIGS. 2 and 3. That is, the thermal packs 20 are separated from the skin of the user 10 by only the material 14 of the garment 12. The arrowed characters Q on FIGS. 2 and 3 are bi-directional (i.e., extend either toward or from the thermal pack 20) because the heat transfer so indicated can be heat flow either from the thermal pack 20 to the user 10 (i.e., supplying heat to the user), or heat flow from the user to the thermal pack 20 (i.e., cooling the user). The thermal packs 20 are active units which may be chemical reaction powered to either provide heat (i.e., exothermic chemical reaction) to the user 10, or to provide cooling (i.e., endothermic chemical reaction) to the user 10. The thermal packs 20 may provide heating or cooling by mechanisms other than chemical reaction. For example, the thermal packs may be thermoelectric, resistance, reverse Peltier effect, or any other desired form of heating or cooling device.

Importantly, the frontal view of user 10 illustrates that the garment 12 positions the thermal packs 20 over and in alignment along their length with the femoral arteries of the user 10. The femoral arteries are generally indicated by the dashed lines “F” on FIG. 1. Because of the elasticity of the garment 12 (i.e., providing mild compression), the thermal packs 20 are held by garment 12 in effective heat transfer relation with the user's skin, and with the user's blood circulating in the femoral arteries F. In this respect it may be considered important that the thermal packs 20 are elongate and are disposed over and in alignment with the femoral arteries F of the user 10. Consequently, heating or cooling provided by the thermal packs 20 is most effectively provided to either heat or cool the circulating core blood of the user 10 at the femoral arteries F.

Consequently, the thermal assistance garment 12, and thermal packs 20, can be utilized by a user to effectively providing cooling to the user's circulating core blood in conditions of extreme exertion or high temperatures, or while wearing protective apparel, for example. On the other hand, heating thermal packs 20 can be used along with garment 12 to assist the user in maintaining a safe core body temperature under frigid conditions.

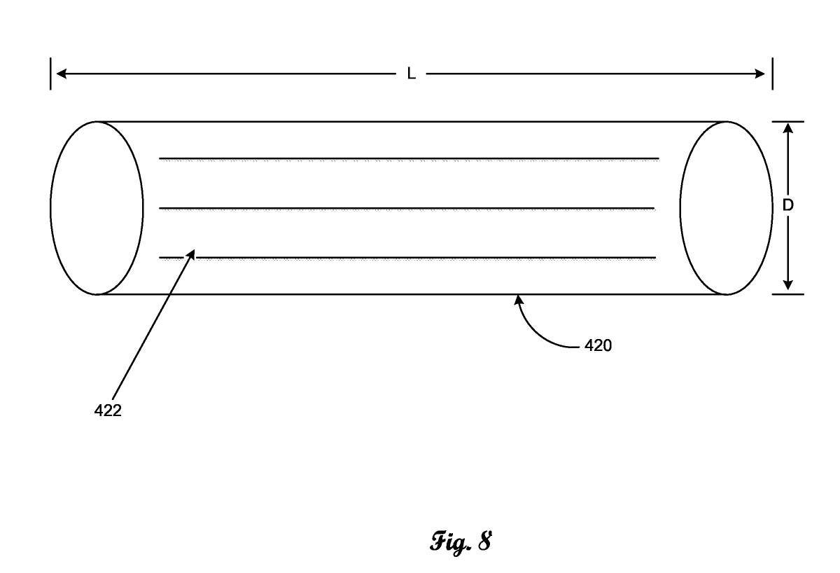

Turning to FIG. 4, an alternative embodiment of a thermal assistance garment is illustrated being worn by a user or wearer 110. Because the garment seen in FIG. 4 is similar in many respects to that of FIGS. 1-3, features which are analogous in structure or function to those of FIGS. 1-3 are referenced on FIG. 4 with the same numeral used above, but increased by one hundred (100). Accordingly, attention to the garment 112 shown in FIG. 4 will reveal that the garment 112 is configured as an elastic compression shirt including a shirt body 112A (only a portion of which is seen in FIG. 4) fashioned of selectively elastic material 114, and a pair of sleeves 1125 also fashioned of selectively elastic material 114 (only one of which—the left sleeve—is seen in FIG. 4).

Accordingly, the shirt configuration of thermal assistance garment 112 seen in FIG. 4 according to this invention includes a pair of elongate pocket features, generally indicated with the arrowed numerals 116. The upper one 116U of this pair of pocket features 116 is disposed over and in alignment along its length with the brachial artery of the user's upper inner arm (indicated by dashed arrow B). On the other hand, the lower one 116L of this pair of pocket features 116 is disposed over and in alignment along its length with the ulnar/radial (i.e., inner wrist) artery of the user 110 (indicated by dashed arrow UL). The pockets 116U and 116L are both disposed in this embodiment on the outside of the shell material 114 of the shirt 112, and each defines a respective upper opening, generally indicated with the arrowed numeral 118. An elongate thermal pack or package 120 (i.e., like the thermal packs 20 discussed above) may be received into either one or both of the pockets 116U and 116L, as will by now be familiar to the reader.

In hot environmental conditions, for example, the wearer 110 may choose to employ a cooling thermal pack in the upper pocket 116U of both arms to assist in preventing overheating of the user's circulating core blood. An additional pair of cooling thermal packs can be used by the user in the lower pockets 116L for this purpose also. However, it will be understood that in cold weather conditions, or other conditions resulting in severe or rapid cooling of the hands, the user may choose to use warming packs in each of the pockets 116U, 116L in order to provide supplemental warming to the user's core blood flow, and especially to provide warming of the blood flow to the user's hands. Such supplemental warming of the blood flow to an extremity (such as the user's hands, via the UL artery, as seen in FIG. 4) can be useful to the user in order to assist in maintaining hand strength and dexterity in cold weather conditions, or in conditions of rapid hand cooling, such as immersion in cold water.

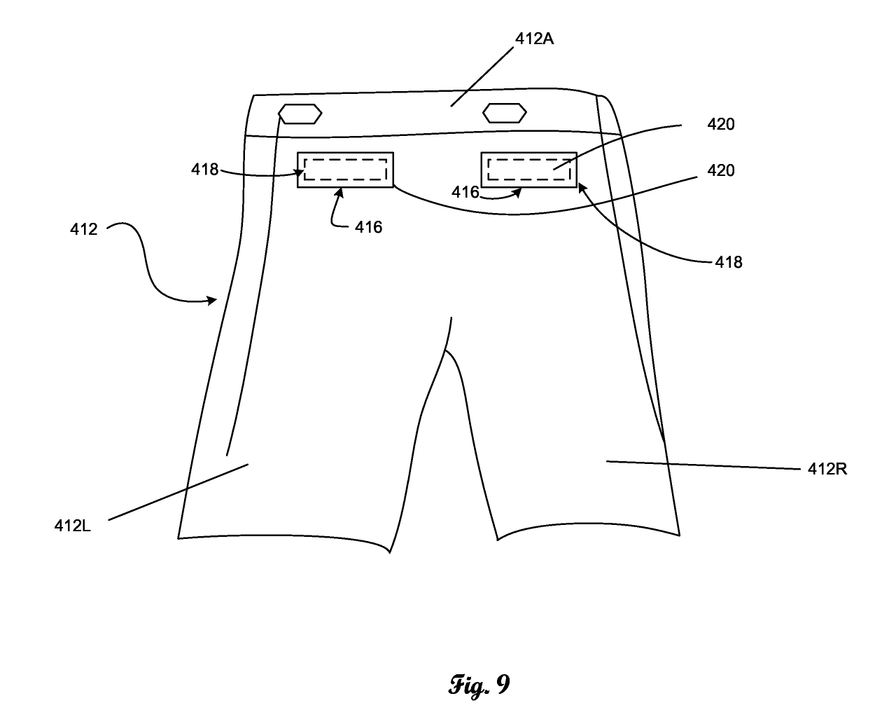

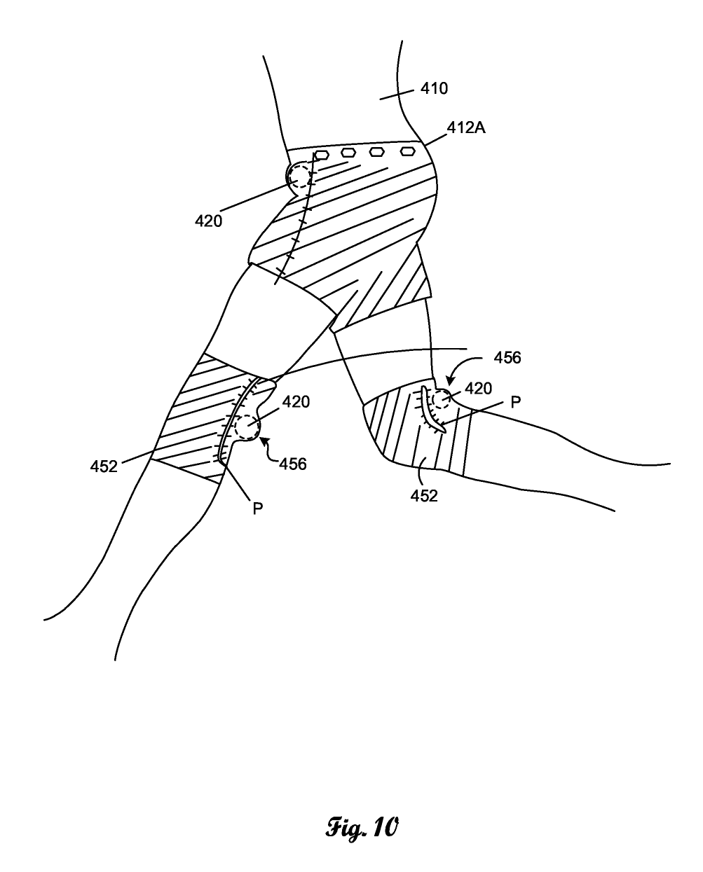

Turning to FIGS. 5 and 6 in conjunction to one another, yet another alternative embodiment of a thermal assistance garment is illustrated being worn by a user or wearer 210. Because the garment seen in FIGS. 5 and 6 is similar in many respects to that of FIGS. 1-3, features which are analogous in structure or function to those of FIGS. 1-3 are referenced on FIGS. 5 and 6 with the same numeral used above, but increased by two hundred (200).

Viewing FIGS. 5 and 6 in conjunction, a thermal assistance garment 212 embodying the present invention is illustrated. The garment 212 in this embodiment is configured as a pair of compression shorts similar in many respects to the embodiment of FIGS. 1-3. Accordingly, the garment 212 includes an elastic outer shell of material 214. The thermal assistance garment 212 according to this invention includes a pair of elongate generally vertically oriented pocket features, generally indicated with the arrowed numerals 216 (only one of which is seen in FIGS. 5 and 6). The pockets 216 are disposed in this embodiment on the inside of the shell material 214, and the shell material 214 outwardly defines merely an upper slit opening 218 opening inwardly to the pocket 216. An elongate thermal pack or package 220 is receivable inwardly via the slit opening 218 to be disposed inside of the garment 212 (i.e., inside of the shell material 214) and within a respective one of the pockets 216, as is indicated in FIGS. 5 and 6.

Importantly, as is seen in FIGS. 5 and 6, the pocket 216 is defined by material 222 which is selectively different from the material 214 from which the shell of the garment 212 is fashioned. The material 222 may be selected to have a heat transfer characteristic (i.e., thermal conductivity, or resistance value) which differs from the material 214. This feature of the embodiment of FIGS. 5 and 6 allows the selection of a material 222 for the pockets 216 which has a higher thermal conductivity than the shell material 214, if such is desirable. On the other hand, the pocket material 222 may also be selected to have a greater insulation value, thus mitigating (or slowing) the heat transfer between the user's core blood flow and the thermal packs 220.

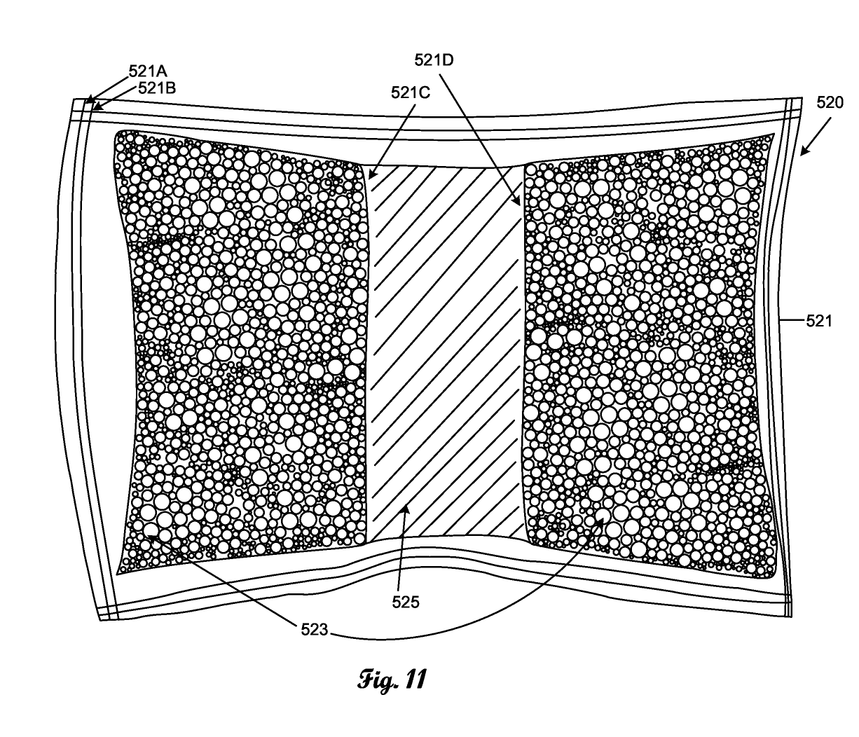

Turning now to FIG. 7 still another alternative embodiment of a thermal assistance device is illustrated. In this case, the device is configured as a band, strap, or wrap that may be worn on a wrist, arm, or leg, for example. Essentially, the device is configured as a flexible structure that may circumscribe and attach to a portion of the human body, such as a wrist, arm, leg, or foot, for example in order to provide a snug fit and mild compression. Because the device seen in FIG. 7 is similar in many respects to those seen in earlier Figures described above, features which are analogous in structure or function to those illustrated and described above are referenced on FIG. 7 with the same numeral used above, but increased by three hundred (300).

Viewing FIG. 7, a thermal assistance device 312 embodying the present invention is illustrated. The device 312 in this embodiment is configured as band, strap, or wrap which may be worn on or about a wrist, arm, leg, or foot, for example. The wearer's wrist, arm, leg, or foot is diagrammatically illustrated in FIG. 7, and is generally indicated in this Figure with the arrowed numeral 310. The device 312 includes a body portion 314 which may be elastic, although such is not essential to operability of the inventive device. That is, the body portion 314 may be configured as a wrap, possibly including hook-and-loop fastening material, which is simply wrapped about a person's arm, wrist, or leg in a selected location. The thermal assistance device 312 includes an elongate pocket, generally indicated with the arrowed numeral 316. The pocket 316 is disposed in this embodiment on the outside of the body 314 (although it may be disposed internally of the device, recalling FIGS. 5 and 6), and the pocket 316 defines an upper slit opening 318. As explained above, an elongate thermal pack or package 320 is receivable into the pocket 316 via the opening 318 to be disposed in abiding heat transfer relation with the skin of the person wearing device 312. As is seen in FIG. 7, the thermal pack 320 is disposed over the superficial exposure of an artery “A” such that an effective heat transfer relation is established between the thermal pack 320, the user's skin, and the underlying artery A. By effecting supplemental heating or cooling to the blood circulating in artery A, the device 312 is effective to assist in heating or cooling the person's core blood, or the blood supply to an extremity, such as a hand or foot.

The inventor has determined that a human's hips and knees are configured differently and move differently than other parts of the human body. As such, the pulse points located at the front of the hips (i.e., femoral artery) and back of the knees (i.e., popliteal artery) maybe superficially exposed due to e.g., movement of these body parts. The inventor has determined that to accommodate this exposure, a different sized and/or shaped heating or cooling device is preferably desired.

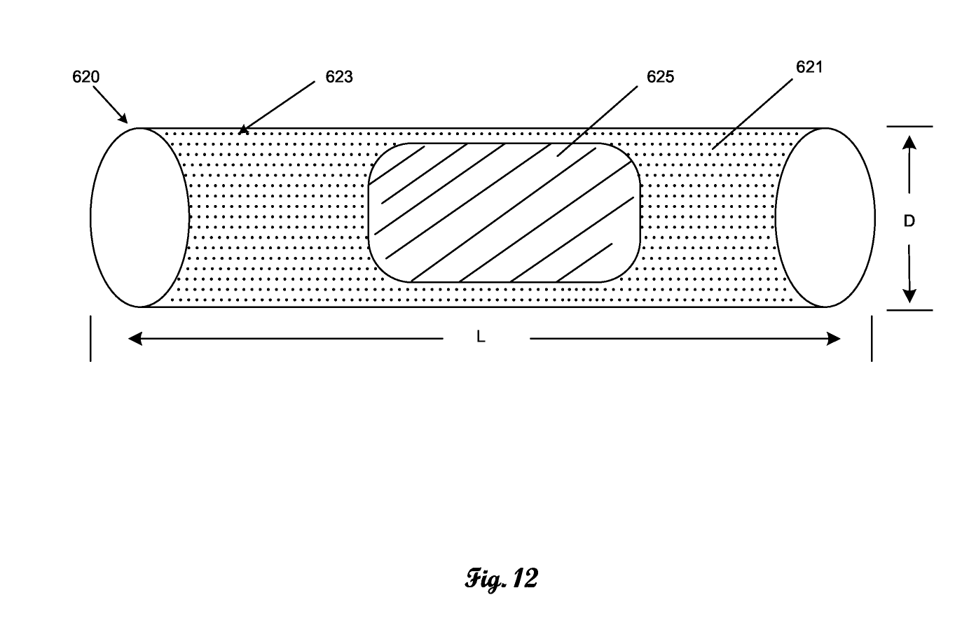

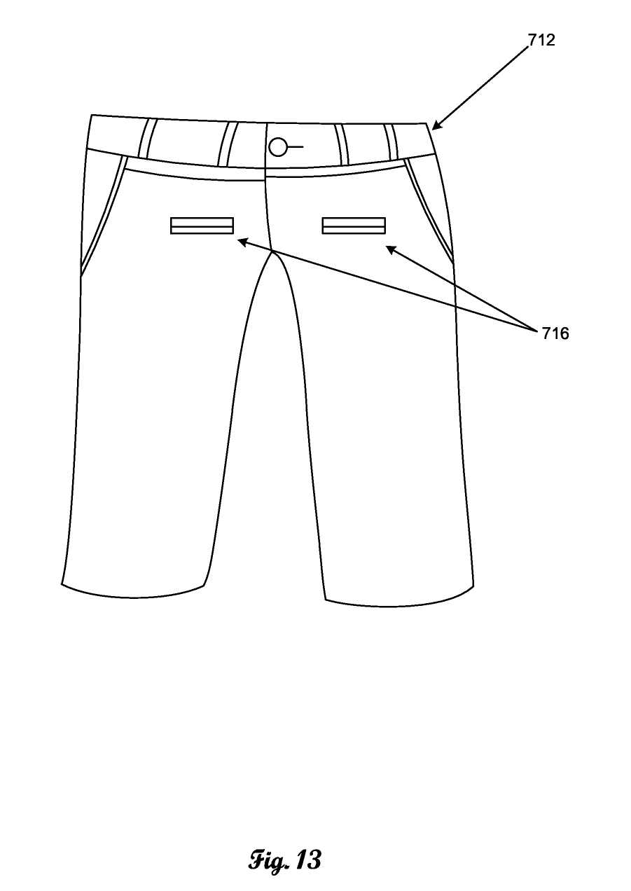

Accordingly, as shown in FIGS. 8-10, a heating or cooling device 420 is provided and configured to address exposure of the femoral artery at the front of the wearer's 410 hips and/or the popliteal artery at the back of the wearer's 410 knee. The illustrated heating or cooling device 420 is configured to be used in a thermal assistance garment 412 having specifically configured pockets 416 for holding the heating or cooling device 420 at the front of the wearer's hips on the femoral artery F (as shown in FIGS. 9 and 10). In addition, the illustrated heating or cooling device 420 is configured to be used in a thermal assistance garment 452 having a specifically configured pocket 456 for holding the heating or cooling device 420 at the back of the wearer's knees on the popliteal artery P (as shown in FIG. 10). As such, the combination of the heating or cooling device 420 with either one or both of the garments 412, 452 comprises a thermal assistance system for heating or cooling the wearer 410 in accordance with the disclosed principles.

The illustrated thermal assistance garment 412 is configured as a pair of compression shorts like garment 12 (FIG. 1) and may be made of the same type of material discussed above. The garment 412 may have a waist band 412a, and left and right leg portions 412L, 412R extending from the waist band 412a. Two pockets 416 are provided for holding heating or cooling devices 420 at appropriate points on the wearer's hips. The heating or cooling devices are inserted through openings 418. As discussed above, the pockets 416 can be disposed on the outside of shell material forming the shorts (i.e., similar to garment 12 of FIGS. 1-3) or on the inside of the shell material forming the shorts (i.e., similar to garment 212 of FIGS. 5 and 6).

The illustrated thermal assistance garment 456 to be worn around one of the wearer's 410 knee is configured as a band, strap or wrap like garment 312 illustrated in FIG. 7. A pocket 456 is provided for holding the heating or cooling device 420 behind the wearer's 410 knee. The heating or cooling devices are inserted through openings (not shown). It should be appreciated that the pocket 456 can be disposed on the outside of shell material forming the a band, strap or wrap or on the inside of the shell material forming the a band, strap or wrap.

The illustrated heating or cooling device 420 is preferably shaped like a “roll of quarters” and is similarly sized. That is, the device 420 has a diameter D approximately the same as the diameter of a quarter (i.e., approximately 0.955 inches) and a length L approximately the same as the length of a roll of quarters (i.e., approximately 2 and ¾ inches). It should be appreciated that the unique shape of the illustrated heating or cooling device 420 allows it to be placed on the body in the desired locations (e.g., joints) without restricting the wearer's 410 movements. While particularly sized for the hips and knees, it should be appreciated that the device 420 may be used at other points of the wearer's body. The housing 422 of the device 420 may comprise plastic, rubber, nylon, spandex, polyurethane, high-density polyethylene (HDPE), nitrile, latex, Tyvek or other waterproof, flexible materials. The device 420 can be manufactured by any process suitable for the material selected, including e.g., vacuum sealing.

The device 420 provides heating or cooling based on the heat transfer principles disclosed above. Accordingly, the device 420 may comprise phase change materials, urea, ammonium nitrate, water and/or any other exothermic or endothermic materials. The heating or cooling device 420 may provide heating or cooling by mechanisms other than chemical reaction. For example, the heating or cooling device 420 may be thermoelectric, resistance, reverse Peltier effect, or any other desired form of heating or cooling device.

The inventor has also determined that other types of heating or cooling devices may be desirable under certain situations. For example, the inventor has recognized that there are challenges associated with phase change materials and the transportation of cooling elements that degrade/discharge as soon as they are removed from the cooling medium. These types of devices are not properly suited for situations in which the properties of the heating or cooling device are not required until a specific point in time and/or where insulation and active cooling mediums are not available (e.g., for military and police operations, hiking, backpacking, search and rescue, disaster relief, HAZMAT, infectious disease response, etc.).

Moreover, there may certain environments (e.g., military or law enforcement situations, to name a few) where it is not desirable, or is even out right dangerous, to have a device the makes noise before, during or after activation. For example, the typical instant activation cold pack includes chemical prills or other substances in a package that must be smashed and shaken to activate the prills/substances. This is a noisy process. In addition, when the pack is moved, the prills make noise before and after activation. Thus, conventional heating or cooling devices are insufficient for situations in which silence is required.

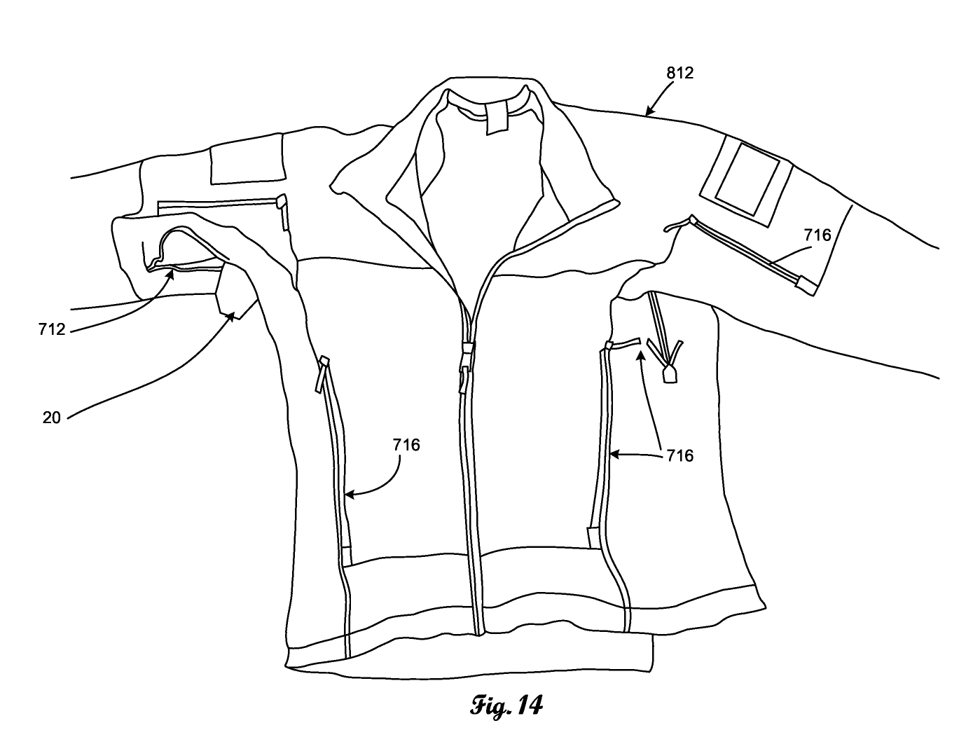

To this end, the FIG. 11 illustrates a heating or cooling device 520 that is vacuum sealed to ensure its silence prior to activation and can be activated by the user at the desired time when the heating or cooling properties are desired. As such, the illustrated device 520 is advantageous over existing heating or cooling devices, particularly for the situations discussed above. It should be appreciated that the illustrated heating or cooling device 520 may be used with any thermal assistance garment disclosed herein. The combination of the heating or cooling device 520 with any garment disclosed herein comprises a thermal assistance system for heating or cooling the wearer in accordance with the disclosed principles.

In the illustrated embodiment, the device 520 comprises a pouch 521 that is double heat-sealed forming seams 521A, 521B to reinforce the pouch 521 against rupture or leakage. The pouch 521 can be made of polyurethane, plastic or any other suitable membrane that is water proof and that can be vacuum sealed. The illustrated device 520 includes a first portion 523 containing a compound (e.g., urea or ammonium nitrate prills) and a second portion 525 comprising a liquid or other substance for activating the compound (e.g., water). In the illustrated embodiment, the second portion 525 is a pouch comprising the activating liquid (e.g., water) that sits within the first portion 523. In another embodiment, the pouch 521 is segmented into three compartments (by seams formed within the pouch at points 521C, 521D) with two compartments filled with the compound (e.g., urea or ammonium nitrate prills) separated by a compartment filled with the activating liquid/substance (e.g., water). It should be appreciated that either configuration is suitable to achieve the benefits disclosed herein. Once the pouch 521 contains the urea or ammonium nitrate prills and the water, the pouch 621 is vacuum sealed.

Hereinafter, it is presumed that the device 520 compound is urea or ammonium nitrate prills that are activated by contact with water. Vacuum sealing prevents the prills from moving around and making noise, which is desirable under any circumstances. In addition, the pouch 521 is sealed with the urea or ammonium nitrate prills in an inactive state because it has not yet been mixed with water. As can be appreciated, the water/urea or ammonium nitrate reaction is critical to the function of the heating or cooling device 520. The reaction is triggered by applying a force to the pouch 521 to rupture the inner water pouch so that the water spills out of the inner pouch and into the prills. The vacuum sealed outer pouch remains intact to contain the now mixed solution. At this point, the device 520 is in its active state providing heating or cooling in accordance with the principles disclosed herein.

FIG. 12 illustrates another heating or cooling device 620 that is vacuum sealed to ensure its silence prior to activation and can be activated by the user at the desired time when the heating or cooling properties are desired. The illustrated device 620 is preferably shaped like a “roll of quarters” and is similarly sized. That is, the device 620 has a diameter D approximately the same as the diameter of a quarter (i.e., approximately 0.955 inches) and a length L approximately the same as the length of a roll of quarters (i.e., approximately 2 and ¾ inches). It should be appreciated that the unique shape of the illustrated heating or cooling device 620 allows it to be placed on the body in the desired locations (e.g., joints) without restricting the wearer's movements.

The illustrated device 620 comprises a pouch 621 that is double heat-sealed to reinforce the pouch 621 against rupture or leakage. The pouch 621 can be made of polyurethane, plastic or any other suitable membrane that is water proof and that can be vacuum sealed. The illustrated device 620 includes a first portion 623 containing a compound (e.g., urea or ammonium nitrate prills) and a second portion 625 comprising a liquid/substance (e.g., water) for activating the compound. In the illustrated embodiment, the second portion 625 is a pouch comprising the liquid (e.g., water) that sits within the first portion 623. In another embodiment, the pouch 621 may be segmented into three compartments (by seams formed within the pouch) with two compartments filled with the compound (e.g., urea or ammonium nitrate prills) separated by a compartment filled with the activating liquid/substance (e.g., water). It should be appreciated that either configuration is suitable to achieve the benefits disclosed herein. Once the pouch 621 contains the compound (e.g., urea or ammonium nitrate prills) and the liquid/substance (e.g., water), the pouch 621 is vacuum sealed. The illustrated device 620 may be activated in the same manner as the device 520 illustrated in FIG. 11. It should be appreciated that the illustrated heating or cooling device 620 may be used with any thermal assistance garment disclosed herein. The combination of the heating or cooling device 620 with any garment disclosed herein comprises a thermal assistance system for heating or cooling the wearer in accordance with the disclosed principles.

The inventor has determined that there are a wide variety of professions and activities where both a base layer of clothing and a protective outer layer must be worn to protect the wearer's body from elements, hazards, the environment, etc. The outer layer garment is typically a jacket, coverall, jumpsuit, uniform or some type of body armor. The thermal assistance devices discussed above provide unique base layers for these professions and activities because they have pockets, etc. that can hold heating or cooling elements that provide the benefits discussed herein. If covered by a protective or other outerwear garment, however, it may be difficult to access the pockets of the disclosed thermal assistance devices using the standard openings in outerwear (e.g., underarm vents, front openings or trouser zipper). Thus, if a heating or cooling element needs to replaced or discarded the wearer may be forced to remove or substantially remove the outerwear garment, exposing the wearer to unnecessary danger or other conditions.

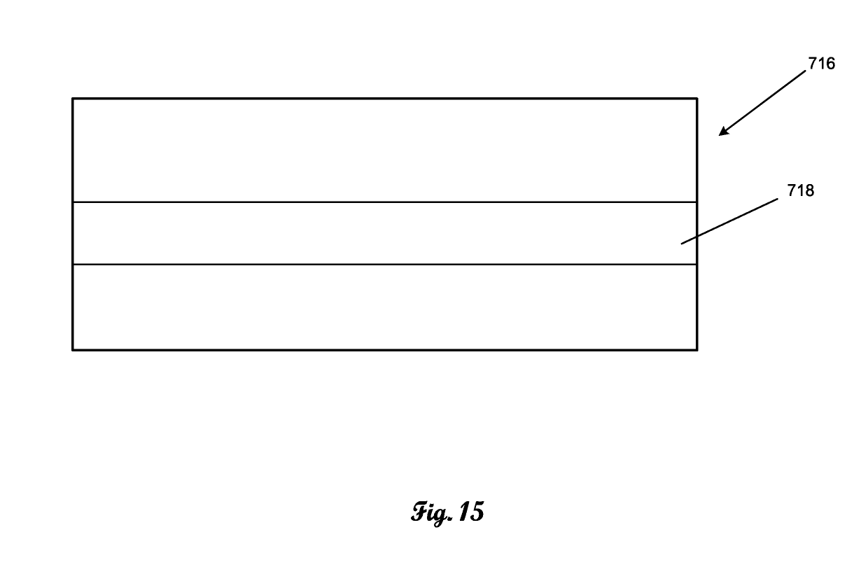

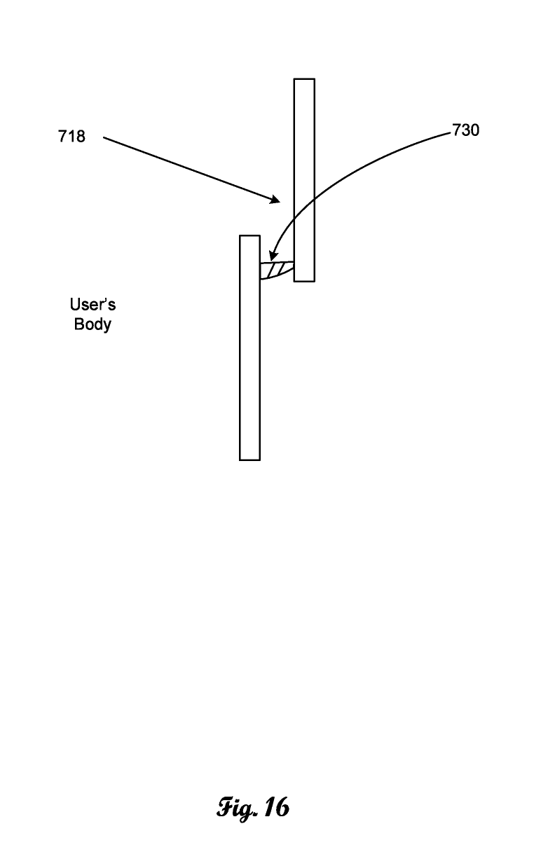

Accordingly, the following embodiment address the need for an improved outerwear garment that has an interface to the underlying pockets of the base layer thermal assistance garment. FIG. 13 illustrates an example embodiment of an outerwear garment 712 to be provided over a thermal assistance device configured as a pair of shorts or pants (e.g., device 12 illustrated in FIGS. 1-3). FIG. 14 illustrates an example embodiment of an outerwear garment 812 to be provided over a thermal assistance device configured as a shirt (e.g., device 112 illustrated in FIG. 4). Both outerwear garments 712, 812 include pocket interfaces 716 providing access to the underlying thermal assistance garment. In desired embodiments, the outerwear garments 712, 812 comprise pocket interfaces 716 for each pocket contained in the underlying thermal assistance garment. It should be appreciated that the combination of the illustrated outerwear garments 712, 812 with the suitable thermal assistance garment disclosed herein comprises a thermal assistance system for heating or cooling the wearer in accordance with the disclosed principles.

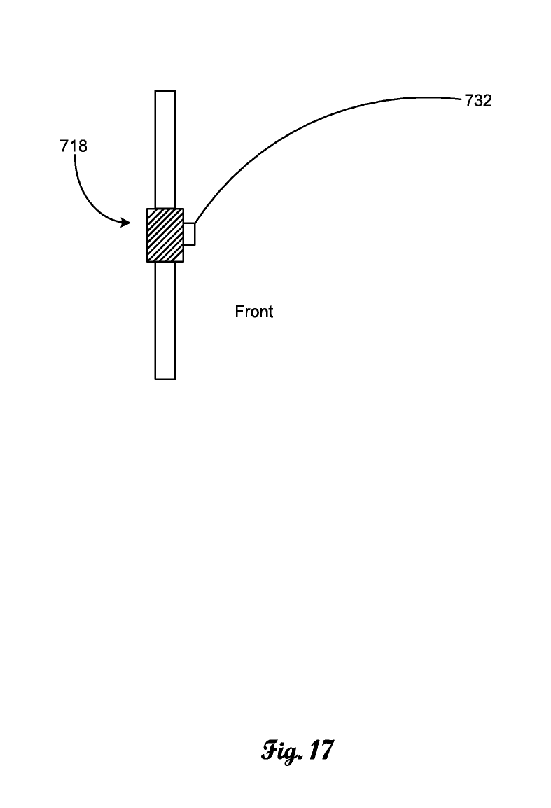

As shown in FIG. 15, the pocket interfaces 716 comprise an opening 718 through the outerwear garment 712, 812. The wearer of the outerwear garment 712, 812 can reach through the opening 718 to get access to the pockets within the base layer thermal assistance device to insert or remove the heating or cooling devices. This is something that is not achievable using standard outerwear garments.

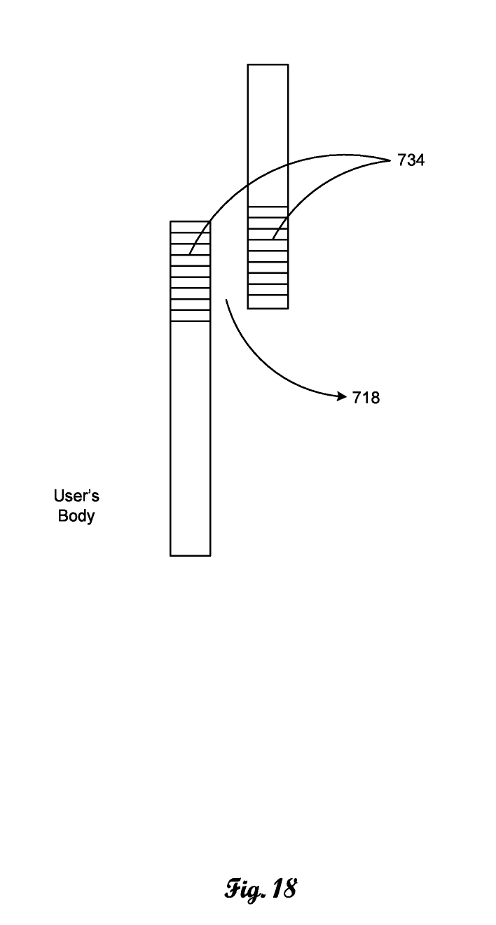

It is desirable that the pocket interfaces 716 be closed when access to the underlying pockets are not necessary. Accordingly, the opening 718 can be closed via a zipper, hook and loop closure, magnet, button, snap, draw cord, or any other type of closure. It should also be appreciated that the opening 718 can be covered by a flap attached to the outerwear garment 712, 812, which may or may not be secured since it covers the opening 718. FIG. 16 illustrates an opening 718 that is closed by a magnet or hook and loop device 730 attached to both ends of the opening 718. FIG. 17 illustrates an opening 718 that is closed by a zipper 732 attached to both ends of the opening 718. FIG. 18 illustrates an opening 718 that is closed by an elastic closure 734 (such as those used in hoods) attached to both ends of the opening 718. It should be appreciated that any type of closure mechanism can be used to close the opening 718. Although the illustrated outerwear garments 712, 812 are shorts and a jacket/shirt, respectively, it should also be appreciated that the disclosed principles apply to an outerwear garment that could be a pair of long pants having access to any heating or cooling device located as discussed herein (i.e., having access to the hips, knees and other areas of the legs) or a band, strap or wrap device particularly suited to cover the band, strap or wrap illustrated in FIG. 7.

Having considered the structure of several alternative embodiments of the thermal assistance garment herein disclosed, illustrated, and described, attention may now be directed more particularly to their method of operation. As will be appreciated viewing the drawing Figures described above, the present inventive garment disposes an elongate heat transfer pack or package 20, 120, 220, 320 420, 520, 620 over one or more selected superficially or near-superficially exposed arteries of the user, which arteries are conveying core blood, or conveying blood to an extremity. The thermal packs are maintained in good heat transfer relationship with the user's skin, and the thermal packs are elongate with their length aligned along the artery to which supplemental heating or cooling is to be provided.

Locations of the human body which are particularly favored for application of the present method are:
the femoral artery (upper thigh, hips, inner groin);
the brachial artery (upper inner arm);
the ulnar/radial artery (inner wrist);
the posterior tibial artery (inner upper ankle);
the dorsalis pedis artery (top of the foot); and
the popliteal artery (back of the knee).

As will be appreciated by those familiar with human anatomy, the first two locations listed above are perhaps the most effective in supplying supplemental heating or cooling to the circulating core blood, although each of these locations can be of assistance in such an effort. The last four locations listed are perhaps of particular interest in providing supplemental heating to the blood supply to an extremity. The ulnar/radial artery is an especially effective location for supplying supplemental blood warming to the hands of a person who must work with high manual dexterity or hand strength in difficult conditions of extreme cold, or in conditions of rapid heat loss from the hands, such as immersion in cold water.

As can be appreciated, the heating or cooling devices and the thermal assistance garments disclosed herein would be suitable for many types of activities, including, but not limited to outdoor activities and other activities including hunting, military, law enforcement, fire/first responder, CBRN, athletics, motorsports, skiing (or other snow related activities), industrial safety, and construction activities, to name a few.

Those skilled in the art will further appreciate that the present invention may be embodied in other specific forms without departing from the spirit or central attributes thereof. For example, the present invention may be embodied in a device or structure to be worn by a human but which is less than or only a portion of a garment, such as a wrist band or leg band, for example. Still alternatively, an arm sleeve or leg sleeve embodying the present invention could be utilized to hold thermal packs in selected locations of the human body consistently with the principles of this invention. Accordingly, because the foregoing description of the present invention discloses only particularly preferred exemplary embodiments of the invention, it is to be understood that other variations are recognized as being within the scope of the present invention. Accordingly, the present invention is not limited to the particular embodiment which has been described in detail herein. Rather, reference should be made to the appended claims to define the scope and content of the present invention.

What is claimed is:

1. A thermal assistance system to be worn by a user, said thermal assistance system comprising:
a thermal assistance garment comprising at least one holder disposed at a selected location of the user's body; and
at least one heating or cooling device configured to fit within the at least one holder,
wherein the at least one holder is configured to maintain the at least one heating or cooling device against the wearer's body at the selected location, and
wherein the at least one heating or cooling device has a shape and size suitable for placement on a front of a hip of or a back of a knee of the user, and/or the at least one heating or cooling device is vacuum sealed in an initial inactive state.

2. The thermal assistance system of claim 1, wherein the at least one heating or cooling device is shaped such that is has a diameter and a length that are substantially the same as a diameter and length of a roll of quarters.

3. The thermal assistance system of claim 2, wherein the at least one heating or cooling device comprises one of plastic, rubber, nylon, spandex, polyurethane, high-density polyethylene (HDPE), nitrile, latex, or Tyvek.

4. The thermal assistance system of claim 2, wherein at least one holder is located at the front of one of the user's hips to cover the user's femoral artery.

5. The thermal assistance system of claim 2, wherein at least one holder is located at the back of one of the user's knees to cover the user's popliteal artery.

6. The thermal assistance system of claim 1, wherein the at least one heating or cooling device is vacuum sealed in the initial inactive state and said heating or cooling device comprises:
a pouch comprising a first portion containing a compound that reacts with a liquid to provide heating or cooling and a second portion comprising the liquid,
wherein the compound and the liquid remain separated in the inactive state, and
wherein a force applied to the pouch causes the compound and liquid to react within the pouch and place the heating or cooling device into an active state.

7. The thermal assistance system of claim 6, wherein the compound is urea or ammonium nitrate prills and the liquid is water.

8. The thermal assistance system of claim 6, wherein the at least one heating or cooling device is shaped such that is has a diameter and a length that are substantially the same as a diameter and length of a roll of quarters.

9. The thermal assistance system of claim 6, wherein the liquid is maintained in a separate pouch within the first portion.

10. The thermal assistance system of claim 1, further comprising an outerwear garment adapted to be placed over the thermal assistance garment, said outerwear garment comprising at least one interface providing access to the at least one holder in the thermal assistance garment.

11. The thermal assistance system of claim 10, wherein the outerwear garment comprises a closing mechanism for closing the at least one interface and preventing access to the at least one holder in the thermal assistance garment.

12. The thermal assistance system of claim 11, wherein the closing mechanism comprises one of a flap, zipper, hook and loop closure, magnet, button, snap, or draw cord mechanism.

13. A heating or cooling device for a thermal assistance garment, said device comprising one or more of a shape and size suitable for placement on a front of a hip of or a back of a knee of a wearer, and/or being vacuum sealed in an initial inactive state.

14. The heating or cooling device of claim 13, wherein the device is shaped such that is has a diameter and a length that are substantially the same as a diameter and length of a roll of quarters.

15. The heating or cooling device of claim 14, wherein the device comprises one of plastic, rubber, nylon, spandex, polyurethane, high-density polyethylene (HDPE), nitrile, latex, or Tyvek.

16. The heating or cooling device of claim 13, wherein the device is vacuum sealed in the initial inactive state and comprises:
a pouch comprising a first portion containing a compound that reacts with water to provide heating or cooling and a second portion comprising water,
wherein the compound and the water remain separated in the inactive state, and
wherein a force applied to the pouch causes the compound and water to react within the pouch and place the heating or cooling device into an active state.

17. The heating or cooling device of claim 16, wherein the compound is urea or ammonium nitrate prills.

18. The heating or cooling device of claim 16, wherein the at least one heating or cooling device is shaped such that is has a diameter and a length that are substantially the same as a diameter and length of a roll of quarters.

19. The heating or cooling device of claim 16, wherein the water is maintained in a separate pouch within the first portion.
